(19) United States
(12) Patent Application Publication
(10) Pub. No.: US 2009/0025728 A1
(43) Pub. Date: Jan. 29, 2009

(54) METHODS AND SYSTEMS FOR MONITORING BREATHING TUBE MOVEMENT

(75) Inventors: Nikolai Aljuri, Revere, MA (US); Rodney C. Perkins, Woodside, CA (US); Jeffrey S. Stuart, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LungPort Corporation c/o Rodney Perkins and Associates, Woodside, CA (US)

(21) Appl. No.: 12/045,845

(22) Filed: Mar. 11, 2008

Related U.S. Application Data
(60) Provisional application No. 60/895,577, filed on Mar. 19, 2007.

(51) Int. Cl. A61M 16/04; A61B 1/267

(52) U.S. Cl. 128/207.14; 600/103

(57) ABSTRACT

Methods and systems are provided for detecting movement of the breathing tube in a surrounding airway of a patient. The breathing tube includes an optical fiber or camera system for producing a video image of the airway from the distal end of the breathing tube. By obtaining a baseline image and comparing said baseline image with successive images, the position of the breathing tube can be automatically monitored and a signal provided when the image differs from the baseline by more than a threshold value.

### CROSS-REFERENCES TO RELATED APPLICATIONS

The present application claims the benefit of provisional U.S. Application No. 60/895,577 (Attorney Docket No. 026560-000200US), filed Mar. 19, 2007, the full disclosure of which is incorporated herein by reference.

### BACKGROUND OF THE INVENTION

1. Field of the Invention

The present invention relates generally to medical methods and apparatus. More particularly, the present invention relates to methods and systems for detecting movement in endotracheal and other breathing tubes after placement in the trachea.

Endotracheal tubes are relatively soft tubular devices which are passed through a patient's nose or mouth and anchored in the trachea by an inflatable cuff. Endotracheal tubes are typically used with ventilators for assisting breathing in patients who are unable to breathe normally or who are being administered anesthetics. Movement of the anchored end of the endotracheal tube after placement can be problematic since it can compromise the ability of the tube to deliver air to the lungs. This is a particular problem with neonates where the trachea may only be 1.5 cm to 2 cm in length and the tube cannot be anchored in the trachea by an inflatable cuff. Very small movements of the neonate's head and neck can easily displace the endotracheal tube, often resulting in extubation or ventilation of only one lung. Such compromised ventilation can create a crisis, with reduced oxygen delivery leading to hypoxemia and possible collapse of the non-ventilated lung which can be fatal if not immediately treated. There are presently no satisfactory methods for automatically monitoring the position of the endotracheal tube within the trachea while nurses and doctors are not present with the patient. Endotracheal tubes are also used during surgical procedures for the delivery of anesthetics. Monitoring of the position of the endotracheal tube is difficult in those circumstances as well.

For these reasons, it would be desirable to provide methods and systems for continuous real time monitoring of the position of endotracheal tubes in patients, particularly in neonates who are very susceptible to minor displacements of the endotracheal tubes. It would be further desirable if such methods and systems could function fully automatically and provide an alarm condition when the endotracheal tube moves past a specified threshold value. It would be particularly desirable if such methods and systems could be implemented with minimal cost and with minimum need for additional equipment and training. The present invention meets at least some of these objectives.

2. Description of the Background Art

U.S. Pat. No. 5,285,778 to Mackin describes an endotracheal tube having optical fibers for illumination and viewing from the distal end. The '778 patent uses the imaging capability to facilitate insertion and allow for visualization without the use of a bronchoscope. The patent further suggests that direct visualization can be used in place of X-rays for determining whether an endotracheal tube has become malpositioned, but does not suggest that it would be possible to continuously or automatically monitor the position to provide an alarm substantially immediately after the endotracheal tube has shifted positions.

### BRIEF SUMMARY OF THE INVENTION

The present invention provides methods and apparatus for the continuous and automatic monitoring of the position of the distal end of a breathing tube, such as an endotracheal tube, when placed in the trachea or elsewhere in the respiratory tract of a patient. The breathing tube will typically be free of balloons, cuffs, and other anchors at its distal end (typically being intended for neonates) and is provided with a capability for imaging in the region around its distal end. The imaging capability may be provided by optical imaging and illumination fibers, as generally described in U.S. Pat. No. 5,285,778, the full disclosure of which is incorporated herein by reference, or by providing a charged coupled device (CCD) camera at or near its distal tip.

Typically, the image will be provided in a digital form comprising a vertical and horizontal array of pixels in a conventional imaging format, such as the four common bit map standards tiff, gif, jpeg, and bmp. The digital image will typically be fed to a video display so that the image can be used for placement, monitoring the condition of the bronchi for the buildup of fluids, or other purposes. Of particular importance to the present invention, the digital image should be refreshed periodically to permit comparison of successive images with an initial or subsequent baseline image which may be taken at or shortly after the initial placement of the breathing tube in the patient or which may be reinitialized at any time after the breathing tube has been placed. Thus, it is possible that the visual image could have at least two modes with a first mode being used for real time image evaluation where the image is refreshed frequently. A second operational mode could also be provided where the image is refreshed over a longer period of time, for example every 10 seconds, where the successive images are then automatically compared on a pixel-by-pixel basis to determine the percentage of pixels which have changed. When the percentage exceeds a minimum threshold, for example 2%, sometimes about 10%, or sometimes greater, an alarm signal will be generated to warn the treatment staff that the breathing tube may have shifted.

The comparison algorithm relies on comparing the baseline pixel matrix with successive pixel matrices, where each pixel is determined to either have changed or to have not changed in a binary comparison. By determining the percentage of those pixels which have changed relative to the total number of pixels, the percentage change versus the baseline can be determined.

### BRIEF DESCRIPTION OF THE DRAWINGS

FIG. 1 illustrates placement of a breathing tube in the trachea of an infant in accordance with the principles of the present invention.

FIG. 1A is a detailed view of the breathing tube of FIG. 1 showing the imaging element at the distal end thereof.

FIG. 2 is a flow diagram of the comparison algorithm of the present invention.

### DETAILED DESCRIPTION OF THE INVENTION

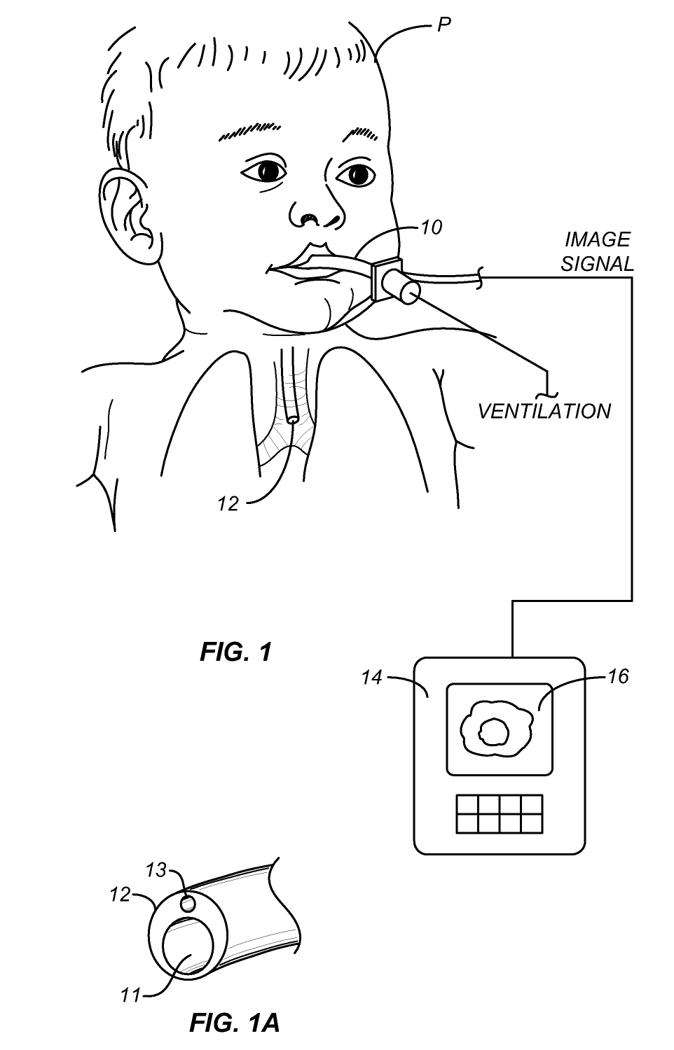

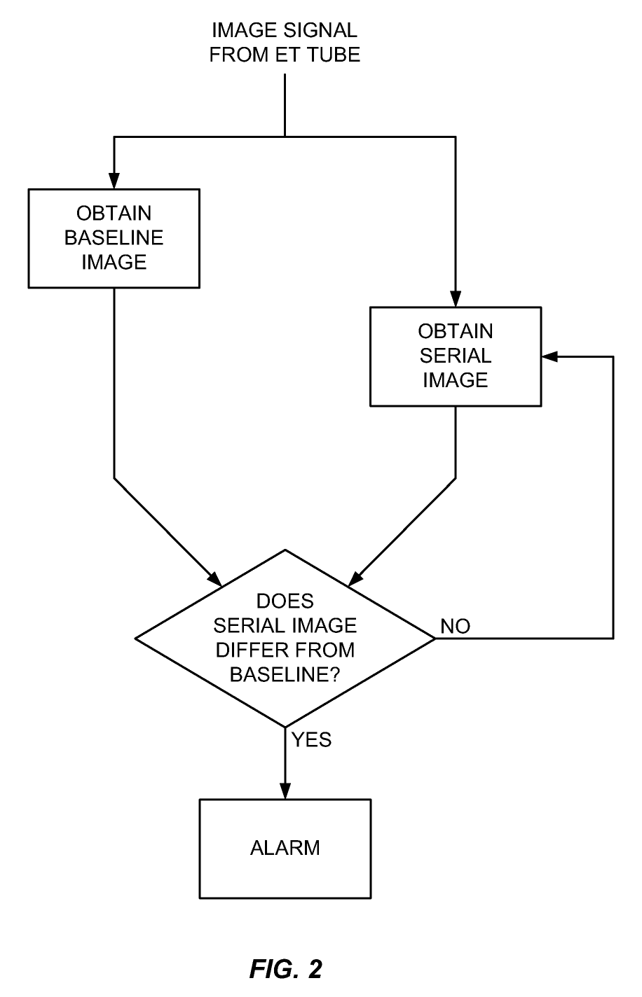

A breathing tube 10, typically an endotracheal tube having a distal end 12, may be placed in an infant or other patient P in a conventional manner, as shown in FIG. 1. The breathing tube 10 has a central passage 11 which will be connected to a ventilator in a conventional manner. In addition, the breathing tube 10 has an imaging element 13 such as a camera or imaging fiber at its distal end 12 (FIG. 1A) which can image the bronchi distal to the trachea T. The breathing tube will usually be connected to an image monitor 14 including a video display 16 and an internal processor (not shown).

In accordance with the principles of the present invention, the processor of the video display 14 will provide for processing of an image signal from the breathing tube, typically the endotracheal tube (ET). First, a baseline image is obtained and stored as a matrix in the memory. After the baseline image is obtained, serial matrix images will be obtained over discrete time differences. Preferably, the discrete time differences will be no greater than 60 seconds, usually no greater than 10 seconds after which time the serial image will be compared with the baseline image. If the serial image does not differ from the baseline by more than a threshold value, typically 2%, sometimes 10%, sometimes greater, then the system will obtain a further serial image at a time within the permitted time interval, again about 60 seconds or less. That further serial image will then be compared against the baseline to determine if the difference exceeds the threshold value. The series of comparisons will be continued, and if one of the comparisons exceeds the baseline difference, then an alarm signal will be provided. Optionally, the base line image can be reestablished if the breathing tube has been intentionally repositioned or if the tube has shifted to a new but acceptable position in the trachea. The alarm signal could trigger a visual, auditory, or other signal. For example, the signal could be forwarded to a monitoring station within the hospital or elsewhere in order to immediately alert the medical staff that there is a possibility that the breathing tube has become malpositioned. The staff can then immediately attend to the patient to determine whether the breathing tube needs to be repositioned or whether the position is still acceptable. After the patient has been treated, the system should be reinitialized to determine a new baseline and the monitoring process may be resumed.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for detecting movement of a breathing tube in a surrounding airway, said method comprising:
generating successive images of the airway from the breathing tube;
comparing at least some of the images with one or more prior images to determine if the image has changed; and
concluding that the breathing tube has moved if the image differs from one or more of the prior images by more than a predetermined amount.

2. A method as in claim 1, wherein generating comprises producing a video image from a camera or lens located on a distal region of the breathing tube.

3. A method as in claim 1, wherein comparing comprises obtaining a baseline image after the breathing tube has been placed and comparing the baseline with at least some of the successive images.

4. A method as in claim 3, wherein images are compared over a time interval no greater than 60 seconds therebetween.

5. A method as in claim 3, wherein comparing comprises determining the percentage of individual pixels in the image which have changed relative to the baseline image.

6. A method as in claim 5, wherein the breathing tube is concluded to have moved when the percentage exceeds about 2%.

7. A system for detecting movement of a breathing tube in an airway of a patient, said system comprising:
a connector for receiving a video output from a camera or lens on a distal portion of the breathing tube;
a processor which compares successive video images from the connector to determine the percentage of individual pixels that have changed; and
an output coupled to the processor which provides a signal when the percentage of individual pictures which have changed exceeds a predetermined percentage.

8. A system as in claim 7, further comprising a breathing tube having a camera or lens near its distal end and a hub for connecting to the connector.

9. A system as in claim 7, wherein the predetermined percentage is about 2%.

10. A system as in claim 7, wherein the images are compared over a time no greater than 60 seconds.
